(19) United States
(12) Patent Application Publication
(10) Pub. No.: US 2019/0076335 A1
(43) Pub. Date: Mar. 14, 2019

(54) ORAL-SURFACE ADMINISTERED PREPARATION FOR THE PREVENTION OF ILLNESSES ACQUIRED VIA THE ORAL CAVITY AND THE PHARYNX

(71) Applicant: IntraMont Technologies, Inc., Hackensack, NJ (US)

(72) Inventors: Joseph G. Montes, Pensacola, FL (US); James Intrater, Hackensack, NJ (US)

(73) Assignee: IntraMont Technologies, Inc., Hackensack, NJ (US)

(21) Appl. No.: 15/701,465

(22) Filed: Sep. 12, 2017

(51) Int. Cl. A61K 6/00; A61K 31/315; A61K 8/27

(52) U.S. Cl. CPC A61K 6/0047; A61Q 11/00; A61K 8/27; A61K 31/315

(57) ABSTRACT

The invention includes a toothpaste or other orally applied product to be used at least once daily to help prevent or inhibit the acquisition of a number of infections by oral or pharyngeal tissues, including the “common” cold. These compositions possess a combination of ingredients not found in other toothpastes or other orally applied products, and these ingredients work together to synergistically interfere with the acquisition of a number of pathogens. The toothpastes will have the usual dentifrice ingredients known to prevent tooth decay, as well as those that reduce halitosis (i.e., bad breath), and prevent gingivitis. In addition, the toothpastes will include one or more of: 1) starch impregnated with a zinc salt, preferably zinc gluconate; 2) corilagin as an individual compound, and not as an extract component; 3) zinc protoporphyrin IX; and 4) one or more other colorless or low-color tannin each as an individual compound, and not as an extract component.

### FIELD OF THE INVENTION

The invention relates to compositions and formulations for preventing or inhibiting the acquisition of infections via the oral cavity and pharynx, as well as methods of using and making these compositions and formulations. Specifically, the invention relates to toothpastes, in the form of a paste or gel; or mouthwashes or oral rinses; or chewable materials, such as a chewing gum or other orally administered preparation for the prevention or inhibition of colds or other upper respiratory and pharyngeal infections, as well as to their use to administering these compositions and formulations to the mouth and/or pharynx to prevent or inhibit of colds or other upper respiratory and pharyngeal infections.

### BACKGROUND OF THE INVENTION

A number of toothpastes and mouthwashes already on the market are known to fight tooth decay (usually through fluoride), to reduce the incidence of gingivitis (usually by the action of triclosan and/or of a zinc salt, such as zinc acetate, zinc gluconate, zinc lactate, or zinc chloride (M. Sanz, et al., Antiplaque and Antigingivitis toothpastes. Monograph Oral Sci (2013) 23:27-44)), to help clean out the mouth of food particles, and to produce “fresh breath.” Thus, while currently available over-the-counter toothpastes address the need for the prevention of dental caries (bacteria-induced tooth decay leading to cavities), gingivitis, amelioration of sensitive teeth, and oral hygiene in general, there is a need in the art for toothpastes and other orally administered preparations that includes ingredients that will enhance the ability of the toothpaste or preparation to prevent or inhibit the acquisition of or reduce the incidence or likelihood of a number of conditions, including the common cold and a host of other pathological conditions caused by pathogenic viruses, bacteria, and fungi, or possibly other conditions (e.g., canker sores, ear infections, etc.). Thus, there is a need in the art for toothpastes and mouthwashes and other formulations that will prevent or inhibit the acquisition of or reduce the incidence or likelihood of a number of infections via the oral cavity and the pharynx, while at the same time function as most toothpastes and mouthwashes in preventing cavities and cleansing the oral cavity.

### SUMMARY OF THE INVENTION

Described here are formulations that when applied to the mouth in the form of one of its embodiments will help prevent or inhibit the acquisition of a large number of possible pathogenic conditions that originate from interaction between a pathogen, such as a virus, bacterium, or fungus, and tissues present in the mouth and/or pharynx. In the preferred embodiment of a toothpaste or gel it can be delivered adequately into the oral and pharyngeal surfaces. By the customary—at least daily—brushing of the teeth, followed by minimal rinsing or merely spitting out of the preparation, typically a toothpaste, the user will be protected from acquiring or have a lower incidence of a number of pathogenic conditions, especially the common cold. Suitable ingredients known in the art will form the base of embodiments of the invention, to which will be added the ingredients of a starch impregnated with a zinc salt, and optionally a dissolved zinc salt (which may or may not be the same zinc compound as the zinc salt impregnated in the starch), and one or more astringent compounds selected from corilagin, another colorless or low-color tannin, and zinc protoporphyrin IX.

In one aspect, provided herein are compositions formulated as a toothpaste or other orally administered preparation for the prevention or inhibition of colds or other upper respiratory and pharyngeal infections, comprising a dentifrice gel or paste comprising (1) a zinc-salt-impregnated starch, and (2) corilagin, or one or more other colorless or low-color tannin (including GALALCOOL®, a low-color tannin extracted from chestnut gallnuts for addition to white wine, sold by Laffort Company based in France), or a combination of corilagin and one or more other colorless or low-color tannin. In some embodiments, the toothpaste or other orally administered preparation further comprises a zinc protoporphyrin IX. In some embodiments, the toothpaste or other orally administered preparation further comprises a free zinc salt.

In another aspect, provided herein are compositions composition formulated as chewing gum, oral rinse, mouth wash, or aerosol for the prevention or inhibition of colds or other upper respiratory and pharyngeal infections, comprising a solvent or other vehicle comprising (1) a zinc-salt-impregnated starch, and (2) corilagin, or one or more other colorless or low-color tannin (e.g., GALALCOOL®) or a combination of corilagin and one or more other colorless or low-color tannin, or a combination of corilagin and or one or more other colorless or low-color tannin, or a combination of corilagin and one or more other colorless or low-color tannin. In some embodiments, the chewing gum, oral rinse, mouth wash, or aerosol further comprises a zinc protoporphyrin IX. In some embodiments, the chewing gum, oral rinse, mouth wash, or aerosol further comprises a free zinc salt.

In another aspect, provided herein are methods of preventing or inhibiting the acquisition of or reduce the incidence or likelihood of a cold or other upper respiratory or pharyngeal infection by administering a composition or formulation described herein to the mouth and/or pharynx of a subject. In yet another aspect provided herein are methods of making a composition or formulation described herein.

Other features and advantages of the present invention will become apparent from the following detailed description examples. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, described herein is a toothpaste, in the form of a paste or gel; or a mouthwash or oral rinse; or a chewable material, such as a chewing gum; or other orally administered preparation that will contain a base preparation with suitable ingredients known in the art, such as a fluoride, a detergent (such as sodium lauryl sulfate), a solvent (such as glycerol and/or propylene glycol), a salt of zinc, an abrasive (such as fumed silica), an antibacterial (such as triclosan), a flavoring agent, including a sweetener, and any other suitable ingredient deemed useful or necessary for the composition or formulation. Added to the base preparation or formulation will be one or more of the following:
(1) starch granules impregnated with a zinc salt, preferably zinc gluconate;
(2) an astringent compound, a tannin, called corilagin;
(3) an astringent compound, a porphyrin, called zinc protoporphyrin IX; and
(4) an astringent compound, a colorless or low-color tannin other than corilagin (e.g., GALALCOOL®).

The above ingredients will produce their effects on preventing or inhibiting the acquisition of a number of pathogens, including viruses that cause colds, such as rhinovirus, including a number of oral bacteria that may be pathogenic, for example, those that may result in gingivitis, and including oral fungi that may produce thrush. Typically, the user when employing a toothbrush to apply a toothpaste described herein will brush with the toothpaste at least once a day, and immediately following brushing will either non-vigorously spit out the preparation only once and/or rinse it out with water.

The toothpaste or other embodiment of the invention will prevent or inhibit the acquisition of or reduce the incidence or likelihood of a number of infections via the oral cavity and the pharynx, while at the same time function as most toothpastes and mouthwashes in preventing cavities and cleansing the oral cavity.

One of the present inventors was commonly beset with the common cold most of his life, typically acquiring 3 to 4 colds per year. However, beginning about twelve years ago he started to correlate lower incidence of his own colds to select ingredients a person might place deliberately in his mouth. Some have been advanced a probable scientific basis, while others have been anecdotally linked to lower incidence of disease, including
1. Salts of zinc;
2. Stannous fluoride or other fluoride;
3. Sodium lauryl sulfate; and
4. Polyphenols, such as tannins.

A number of toothpastes and mouthwashes already on the market are known to fight tooth decay (usually through fluoride), to reduce the incidence of gingivitis (usually by the action of triclosan and/or of a zinc salt, such as zinc acetate, zinc gluconate, zinc lactate, or zinc chloride (M. Sanz, et al., Antiplaque and Antigingivitis toothpastes. Monograph Oral Sci (2013) 23:27-44)), to help clean out the mouth of food particles, and to produce “fresh breath.” However, no toothpaste or mouthwash has the unique combination of ingredients in embodiments of the present invention; in particular, an ingredient in the latter toothpaste provides for the slower release of zinc salts. Zinc salts have been used for many years in a number of toothpastes to prevent gingivitis, but given that zinc salts can also be used to reduce the symptoms of the common cold by apparently reducing the infectivity of the cold virus to mucous membranes, it makes sense to provide for a steadier source of zinc ions by using a method, described below, for rendering zinc salts present in the oral cavity for a longer time period than normally achieved by brushing with current toothpastes. The new toothpaste is typically used at least once per day through brushing with a toothbrush; nonetheless, it can also be used two or more times per day without ill effects or reduction in efficacy. After brushing, the user is to spit out the ingredients from his mouth by no more than one rinse with water, or even without partially or fully rinsing, in order to allow for the adequate retention of residues of the toothpaste.

With regards to the zinc salts, it is now known that zinc ions can prevent the attachment of rhinovirus to cells, and that zinc-containing lozenges can mitigate the symptoms and duration of the common cold (See WedMD. Cold, Flu & Cough Health Center—Zinc for Colds: Lozenges & Nasal Sprays. Available at www.webmd.com/cold-and-flu/cold-guide/zinc-lozenges-cold-remedy; and National Institutes of Health Office of Dietary Supplements. Zinc Fact Sheet for Health Professionals. Available at ods.od.nih.gov/factsheets/Zinc-HealthProfessional). However, the observation that one-and-a-half days of not exposing himself to such ingredients resulted in again the acquiring a common cold by one of the inventors, suggested that extension of the residing time of the anti-colds ingredients in the mouth and pharynx could enhance the effectiveness of cold prevention by zinc compounds. Thus, one of the inventors came up with a method for accomplishing the latter goal of the toothpaste. Focusing currently on the zinc-based ingredient(s), the method includes the steps of: combining a zinc salt with starch in an aqueous solution and allowing the solution to evaporate completely, leaving behind a cake of starch matrix in which the zinc salt is embedded; the preferred salt is zinc gluconate because it tightly comingles chemo-physically with the starch, given the commonality in molecular structure between the gluconate ion of the salt and the glucose molecule that is the monomer constituting the starch (a polymer of glucose). The cake is then ground up to a powder before adding it to the toothpaste. It should be noted that zinc seems to have effects on other common viruses, such as respiratory syncytial virus (Effect of Zinc Salts on Respiratory Syncytial Virus Replication—NCBI. Available at www.ncbi.nlm.nih.gov/pmc/articles/PMC353050), and thus extending the residing time of zinc salts could produce other health benefits of the toothpaste. Release of the zinc from the zinc-salt impregnated starch will be increased by amylase, an enzyme present in saliva and which breaks down starch.

There may be a host of infectious viruses whose ability to infect individuals can be hampered by a toothpaste that contains zinc and applies the latter to the oral cavity, gums, and pharynx. In addition, zinc salts are known to have anti-bacterial and anti-fungal properties as well, that along with fluoride salts, detergents, and other components will also help reduce the frequency of acquiring fungal and bacterial infections. Thus, embodiments of the invention address a broad spectrum of pathologies acquired local to the oral cavity and the pharynx.

A property of zinc salts that correlates with their ability to prevent acquisition of colds (rhinoviruses and other viruses) is that of astringency (see www.britannica.com/science/astringent. Accessed Sep. 14, 2016). Zinc belongs in the class of astringents called “metallic astringents”, namely those that cause coagulation effects on the surface layers of cells. This would suggest that coagulation of proteins is involved in prevention or inhibition of acquisition of colds. Given that protein coats called capsids surround all viruses, coagulation of the proteins in the coats would radically interfere with the attachment of the virions (individual virus particles) to their cell hosts; this would apply specifically to the host cells superficially exposed to astringents at their surfaces. However, many viruses also have an “envelope” of lipid surrounding the protein coat, so that coagulation effects of an astringent may be interfered with; furthermore, such an envelope is believed to interfere with immune defenses by interfering with protein (virus)-to-protein (antibody) binding. However, toothpastes and other oral and pharyngeal preparations contain surfactants of high efficiency, such as sodium lauryl sulfate, thus helping to solubilize the lipid envelopes of the viruses and thus resulting in the exposure of the protein capsid to the astringent in the preparation. Thus, it appears that a detergent environment is a co-factor in the successful prevention by an astringent (including a zinc salt) of the acquisition of an infection from an enveloped virus. As most colds are caused by rhinoviruses, and they are not enveloped viruses, the need for a detergent co-factor to prevent acquiring them in the pharynx is probably not significant. Nonetheless, embodiments of the invention are intended for the prevention or inhibition of acquisition of not just cold viruses, but also other viruses acquired in the mouth or pharynx, and many of them are enveloped viruses. Among enveloped viruses that embodiments of the invention may prevent or inhibit are the following: herpes simplex I (oral herpes); varicella virus (chicken pox); influenza virus; flavivirus (hepatitis C); respiratory syncytial virus, among others (medimoon.com/2014/03/list-of-some-common-viral-diseases-and-their-treatment). It should be noted that the point of initial acquisition of virus is not necessarily representative of the virus's final distribution and symptomatology. For example, one may acquire hepatitis C through the pharynx, but the primary focus of the disease is the liver. By the same token, an inner ear infection (otitis media) can be secondarily acquired from a viral or bacterial infection acquired first in the pharynx.

Thus, the property of astringency is linked to stopping the acquisition of viruses by cells of mucous membranes. In embodiments of the present invention, an isolated tannin called corilagin is an ingredient that is used to prevent the acquisition of pathological conditions caused by microbes, including viruses and bacteria, via the oral cavity and pharynx. This is at least in part because of its known astringent properties. It will act as a coagulation agent as in the case of zinc. However, unlike zinc alone, it will have an affinity for mucus secretions not expected with the ionic form of zinc (from zinc salts). Thus, it may be able to adhere for longer times to certain mouth and pharyngeal surfaces. Furthermore, it has a beneficial anti-inflammatory property as indicated in at least one study (P. Buzzini, et al., Antimicrobial and Antiviral Activity of Hydrolysable Tannins. Mini-Reviews in Medicinal Chemistry (2008) 8:1179-1187; Yuan-Jin Guo et al., Effect of Corilagin on Anti-inflammation in HSV-1 Encephalitis and HSV-1 Infected Microglias. European Journal of Pharmacology (2010) 635:79-86; J. Luoqi, et al., A Potential Anti-tumor Herbal Medicine, Corilagin, Inhibits Ovarian Cancer Cell Growth Through Blocking the TGF-β Signaling Pathways. BMC Complementary and Alternative Medicine (2013) 13:33; N. Bismelah, et al., Journal of Medicinal Plants Studies (2016) 4:18-23; A Gupta, et al., Phytochemistry and Pharmacological Activities of Haritaki—A Review. Journal of Pharmacy Research (2010) 3:417-424).While an extract of the Indian gooseberry has been proposed as a toothpaste ingredient (P Potduang, et al., The Development of Phyllanthus emblica and Zanthoxylum limonella Toothpaste. The 6th International Conference on Natural Products for Health and Beauty (NATPRO6), Jan. 21-23, 2016), and that extract has corilagin as one of its constituents, many other constituents exist in that extract. Furthermore, in that case, corilagin was not singled out as the active ingredient in the preparation nor was its mode of action as an antimicrobial and anti-inflammatory revealed. It should be noted also that the preferred concentration of corilagin to be used in embodiments of the present invention are 0.03% by weight, which is much lower than the extract concentration maximum of 0.2% by weight in the toothpaste with the extract. However, a much higher concentration of corilagin may prove optimal in the future, and as per claims of this patent, it may be as high as 10% of the toothpaste composition by weight, which is very much higher than 0.2%. In such a case, the concentration of pure corilagin in embodiments of the present invention will exceed that in the Indian gooseberry toothpaste by many factors.

Other astringent compounds, namely tannins that are colorless or of low color, would be alternative ingredients to corilagin, although one or more such tannins may also be used together with the latter to extend efficacy of the dentifrice. Tannins, polyphenolic compounds of which corilagin is one example, are found at relatively high concentration in many edible plants. They are astringent and thus typically result in the precipitation of proteins, and furthermore, given that most viruses have a protein capsid (shell) interfacing with the external environment, this could be of central importance in removing viruses from circulation in the mouth and throat (A Manual of Materia Medica and Pharmacology, p. 282. D M R Culbreck. Lea and Febiger, Philadelphia, 1927). In addition, this would suggest an ability, like that of zinc, to interfere with the attachment of certain viruses to cells of mucous membranes, while its very low water solubility suggests that it may preferentially sequester at different locations in the mouth, specifically those not easily wetted, unlike zinc salts. Also, it has been reported that polyphenols, such as the flavonoid compound quercetin, have antibacterial properties, hence possessing other potential benefits to oral health, and not just as a potential anti-viral agent (T P T Cushnie and A J Lamb, Antimicrobial Activity of Flavonoids. International Journal of Antimicrobial Agents (2005) 26:343). Other tannins beside corilagin will be considered as active ingredients in certain embodiments of the invention. However, as tannins typically are dark-colored compounds, only certain tannins will be considered as potential co-ingredients in certain embodiments of the invention; tannins typically stain tooth enamel, so it is important that only colorless or low-color tannins be used in certain embodiments of the invention. They may also stain toothbrush bristles, producing another psychologically objectionable result. Corilagin and/or other tannin or tannins used in embodiments of the invention will be compound(s) displaying potentially synergistic efficacy with the other component ingredients of embodiments of the invention. As for concerns on the toxicology of tannins, the Select Committee on GRAS substances has weighed in on the potential toxicity of tannic acid (as a hydrolysable gallotannin prototype) as follows (from https://www.fda.gov/Food/IngredientsPackagingLabeling/GRAS/SCOGS/ucm261485.htm, accessed Aug. 23, 2017): “The ingredients in the compositions of embodiments of the invention are at concentrations known not to be toxic, even if the product is inadvertently completely swallowed every time it is used. The Select Committee, therefore, in the light of the foregoing concludes that:
There is no evidence in the available information on tannic acid (hydrolyzable gallotannins) that demonstrates or suggests reasonable grounds to suspect a hazard to the public when it is used at levels that are now current and in the manner now practiced. However, it is not possible to determine, without additional data, whether a significant increase in consumption would constitute a dietary hazard.”

Finally, another ingredient, zinc protoporphyrin IX (RF Labbe, et al., Zinc Protoporphyrin: A Metabolite with a Mission. Clinical Chemistry (1999) 45:2060-2072), may be a constituent of the orally administered product. In addition to being yet another zinc source, in this case of longer persistence, and an astringent in its own right, it is a known carrier of divalent ions (such as zinc ion), thus creating a more persistent zinc source that assists in conveying zinc to more extensive areas of the mouth and pharynx, and itself prolonging astringency in the latter areas. Zinc salts, being water soluble, will more easily wash out of the mouth and areas continuous with and beyond the oral cavity.

Because each ingredient has different physical and chemical properties, however, notwithstanding their commonality of astringency, a synergistic effect will result because of their differences in structural stability, affinities for different surfaces, and tendencies to associate and adhere to different secretions.

Although currently available over-the-counter toothpastes address the need for the prevention of dental caries (bacteria-induced tooth decay leading to cavities), gingivitis, amelioration of sensitive teeth, and oral hygiene in general, the novel toothpastes described here may work in a synergistic mode by combining multiple ingredients. It can be expected that since the surface affinity of each ingredient differs according to specific tissue (e.g., gums vs. mucous membranes of the pharynx vs. tooth enamel), that the different ingredients will work to create a more thorough exposure of different tissues to the effective ingredients; furthermore, different strains of bacteria and viruses may differ substantially in susceptibility to the different ingredients, so that diversity in the ingredients should have more effect than the use at simply the highest safe concentration of a single ingredient known to be effective. The ingredients in the novel toothpaste will be present in a unique combination that includes ingredients that will enhance the ability of the toothpaste to prevent the acquisition of a number of conditions, including the common cold and a host of other pathological conditions caused by pathogenic viruses, bacteria, and fungi, or possibly other conditions (e.g., canker sores, ear infections, etc.). The consumer of the toothpaste should preferably need only brush his teeth once per day to attain desired results.

The following examples are presented in order to more fully illustrate the preferred embodiments of the invention. They should in no way be construed, however, as limiting the broad scope of the invention.

Embodiments of the invention include:
1. A composition formulated as a toothpaste or other orally administered preparation for the prevention or inhibition of colds or other upper respiratory and pharyngeal infections, comprising a dentifrice gel or paste comprising (1) a zinc-salt-impregnated starch, and (2) corilagin, one or more other colorless or low-color tannin, or a combination of corilagin and one or more other colorless or low-color tannin.
2. The composition of embodiment 1, wherein the zinc salt is an organic zinc salt.
3. The composition of embodiment 2, wherein the organic zinc salt is selected from the group consisting of zinc gluconate, zinc lactate, zinc acetate, and combinations thereof.
4. The composition of embodiment 3, wherein the organic zinc salt is zinc gluconate.
5. The composition of embodiment 1, wherein the zinc salt is an inorganic zinc salt.
6. The composition of embodiment 5, wherein the inorganic zinc salt is selected from the group consisting of zinc chloride, zinc sulphate, zinc carbonate, and combinations thereof.
7. The composition of any one of embodiments 1 to 6, wherein the starch is an amylopectin.
8. The composition of any one of embodiments 1 to 6, wherein the starch is of botanical origin.
9. The composition of any one of embodiments 1 to 6, wherein the starch is a synthetic starch.
10. The composition of any one of embodiments 1 to 10, where the zinc-salt-impregnated starch is 2% -10% by weight of the composition, and the zinc salt to starch ratio ranges from 1:30 to 1:2 by weight.
11. The composition of embodiment 10, where the where the zinc-salt-impregnated starch is 5% by weight of the composition, and the zinc salt to starch ratio is 1:10 by weight.
12. The composition of any one of embodiments 1 to 11, wherein the corilagin is 0.01% to 10% by weight of the composition.
13. The composition of embodiment 12, wherein the corilagin is 0.03% by weight of the composition.
14. The composition of any one of embodiments 1 to 13, wherein another colorless or low-color tannin, or each other colorless or low-color tannin, is 0.01% to 10% by weight of the composition.
15. The composition of embodiment 14, wherein one or more other colorless or low-color tannin is each 0.03% by weight of the composition.
16. The composition of embodiments 14 and 15 wherein the concentration of corilagin is zero.
17. The composition of any one of embodiments 1 to 11, wherein the composition comprises a combination of corilagin and one or more other colorless or low-color tannin.
18. The composition of embodiment 17, wherein the corilagin is 0.01% to 10% by weight and that of another or each other colorless or low-color tannin is 0.01% to 10% by weight of the composition, respectively.
19. The composition of embodiment 17, wherein the corilagin is 0.03% by weight and that of another or each other colorless or low-color tannin is 0.03% by weight of the composition, respectively.
20. The composition of any one of embodiments 1 to 18, further comprising zinc protoporphyrin IX.
21. The composition of embodiment 19, wherein the zinc protoporphyrin IX ranges from 0.1% to 5% by weight of the composition.
22. The composition of embodiment 20, wherein the zinc protoporphyrin IX is 0.5% by weight of the composition.
23. The composition of any one of embodiments 1 to 21, further comprising a free zinc salt.
24. The composition of embodiment 22, wherein the free zinc salt ranges from 0.1% to 0.5% by weight of the composition.
25. The composition of embodiment 23, wherein the free zinc salt is 0.4% by weight of the composition.
26. The composition of any one of embodiments 1 to 24, further comprising a fluoride compound.
27. A composition formulated as chewing gum, oral rinse, mouth wash, or aerosol for the prevention or inhibition of colds or other upper respiratory and pharyngeal infections, comprising a solvent or other vehicle comprising (1) a zinc-salt-impregnated starch, and (2) corilagin, and one or more other colorless or low-color tannin or a combination of corilagin and one or more other colorless or low-color tannin.
28. The composition of embodiment 26, wherein the zinc salt is an organic zinc salt.
29. The composition of embodiment 27, wherein the organic zinc salt is selected from the group consisting of zinc gluconate, zinc lactate, zinc acetate, and combinations thereof.
30. The composition of embodiment 28, wherein the organic zinc salt is zinc gluconate.
31. The composition of embodiment 26, wherein the zinc salt is an inorganic zinc salt.
32. The composition of embodiment 39, wherein the inorganic zinc salt is selected from the group consisting of zinc chloride, zinc sulphate, zinc carbonate, and combinations thereof.
33. The composition of any one of embodiments 26 to 31, wherein the starch is an amylopectin.
34. The composition of any one of embodiments 26 to 31, wherein the starch is of botanical origin.
35. The composition of any one of embodiments 26 to 31, wherein the starch is a synthetic starch.
36. The composition of any one of embodiments 26 to 34, where the zinc-salt-impregnated starch is 2% -10% by weight of the composition, and the zinc salt to starch ratio ranges from 1:30 to 1:2 by weight.
37. The composition of embodiment 35, where the where the zinc-salt-impregnated starch is 5% by weight of the composition, and the zinc salt to starch ratio is 1:10 by weight.
38. The composition of any one of embodiments 26 to 36, wherein the corilagin is 0.01% to 10% by weight of the composition.
39. The composition of embodiment 37, wherein the corilagin is 0.03% by weight of the composition.
40. The composition of any one of embodiments 26 to 38, wherein the concentration of one or more other colorless or low-color tannin is each 0.01% to 10% by weight of the composition.
41. The composition of embodiment 39, wherein one or more other colorless or low-color tannin is each 0.01% by weight of the composition.
42. The composition of any one of embodiments 26 to 36, wherein the composition comprises a combination of corilagin and one or more other colorless or low-color tannin.
43. The composition of embodiment 41, wherein the corilagin is 0.01% to 10% by weight and that of one or more other colorless or low-color tannin is each 0.01% to 10% by weight of the composition, respectively.
44. The composition of embodiment 42, wherein the corilagin is 0.03% by weight and that of one or more other colorless or low-color tannin is each 0.03% by weight of the composition, respectively.
45. The composition of any one of embodiments 26 to 43, further comprising zinc protoporphyrin IX.
46. The composition of embodiment 44, wherein the zinc protoporphyrin IX ranges from 0.1% to 5% by weight of the composition.
47. The composition of embodiment 45, wherein the zinc protoporphyrin IX is 0.5% by weight of the composition.
48. The composition of any one of embodiments 26 to 46, further comprising a free zinc salt.
49. The composition of embodiment 47, wherein the free zinc salt ranges from 0.1% to 0.5% by weight of the composition.
50. The composition of embodiment 48, wherein the free zinc salt is 0.4% by weight of the composition.
51. The composition of any one of embodiments 26 to 49, further comprising a fluoride compound.
52. The composition of any one of embodiments 1 to 25, further comprising a malleable or squeezable tube in which the composition is stored.
53. The composition of any one of embodiments 1 to 25 or 51, wherein the composition is formulated as a toothpaste and as a translucent gel or as a paste.
54. A method of preventing or inhibiting the acquisition of a cold or other upper respiratory or pharyngeal infection comprising administering to a subject the toothpaste of any one of embodiments 1 to 25 or 51 to 52.
55. A method of reducing the incidence or likelihood of a cold or other upper respiratory or pharyngeal infection comprising administering to a subject the toothpaste of any one of embodiments 1 to 25 or 51 to 52.
56. The method of embodiments 53 or 54, wherein the step of administering comprises brushing of the subject's teeth.
57. The composition of any one of embodiments 26 to 49, wherein the composition is formulated as an oral rinse or mouthwash.
58. A method of preventing or inhibiting the acquisition of a cold or other upper respiratory or pharyngeal infection comprising administering to a subject the oral rinse or mouthwash of embodiment 56.
59. A method of reducing the incidence or likelihood of a cold or other upper respiratory or pharyngeal infection comprising administering to a subject the oral rinse or mouthwash of embodiment 56.
60. The method of embodiments 57 or 58, wherein the step of administering comprises rinsing out the subject's mouth with the oral rinse or mouthwash.
61. The composition of any one of embodiments 26 to 49, wherein the composition is formulated as a chewing gum.
62. A method of preventing or inhibiting the acquisition of a cold or other upper respiratory or pharyngeal infection comprising administering to a subject the chewing gum of embodiment 60.
63. A method of reducing the incidence or likelihood of a cold or other upper respiratory or pharyngeal infection comprising administering to a subject the chewing gum of embodiment 60.
64. The method of embodiments 61 or 62, wherein the step of administering comprises chewing the chewing gum.
65. The composition of any one of embodiments 26 to 49, wherein the composition is formulated as an aerosol to be sprayed into the mouth and/or pharynx.
66. A method of preventing or inhibiting the acquisition of a cold or other upper respiratory or pharyngeal infection comprising administering to a subject the aerosol of embodiment 64.
67. A method of reducing the incidence or likelihood of a cold or other upper respiratory or pharyngeal infection comprising administering to a subject the oral rinse or mouthwash of embodiment 64.
68. The method of embodiments 65 or 66, wherein the step of administering comprises spraying the aerosol into the mouth and/or pharynx of the subject.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1. A composition formulated as a toothpaste or other orally administered preparation for the prevention or inhibition of colds or other upper respiratory and pharyngeal infections, comprising a dentifrice gel or paste comprising (1) a zinc-salt-impregnated starch, and (2) corilagin, or another one or more colorless or low-color tannin, or a combination of corilagin and one or more other colorless or low-color tannin.

2. The composition of claim 1, wherein the zinc salt is an organic zinc salt selected from the group consisting of zinc gluconate, zinc lactate, zinc acetate, and combinations thereof.

3. The composition of claim 2, wherein the organic zinc salt is zinc gluconate.

4. The composition of claim 1, wherein the zinc salt is an inorganic zinc salt selected from the group consisting of zinc chloride, zinc sulphate, zinc carbonate, and combinations thereof.

5. The composition of claim 1, wherein the starch is an amylopectin.

6. The composition of claim 1, where the zinc-salt-impregnated starch is 2% -10% by weight of the composition, and the zinc salt to starch ratio ranges from 1:30 to 1:2 by weight.

7. The composition of claim 1, wherein the corilagin is 0.01% to 10% by weight of the composition.

8. The composition of claim 1, wherein one or more other colorless or low-color tannin is each is 0.01% to 10% by weight of the composition.

9. The composition of claim 1, further comprising zinc protoporphyrin IX and wherein the zinc protoporphyrin IX ranges from 0.1% to 5% by weight of the composition.

10. The composition of claim 1, further comprising a free zinc salt and wherein the free zinc salt ranges from 0.1% to 0.5% by weight of the composition.

11. The composition of claim 1, further comprising a fluoride compound.

12. The composition of claim 1, further comprising wherein the composition is stored in a malleable or squeezable tube.

13. A composition formulated as chewing gum, oral rinse, mouth wash, or aerosol for the prevention or inhibition of colds or other upper respiratory and pharyngeal infections, comprising a solvent or other vehicle comprising (1) a zinc-salt-impregnated starch, and (2) corilagin, or one or more other colorless or low-color tannin, or a combination of corilagin and one or more other colorless or low-color tannin.

14. The composition of claim 13, wherein the zinc salt is an organic zinc salt selected from the group consisting of zinc gluconate, zinc lactate, zinc acetate, and combinations thereof.

15. The composition of claim 13, where the zinc-salt-impregnated starch is 2%-10% by weight of the composition, and the zinc salt to starch ratio ranges from 1:30 to 1:2 by weight.

16. The composition of claim 13, wherein the corilagin is 0.01% to 10% by weight of the composition.

17. The composition of claim 13, wherein one or more other colorless or low-color tannin is each 0.01% to 5% by weight of the composition.

18. The composition of claim 13, further comprising zinc protoporphyrin IX and wherein the zinc protoporphyrin IX ranges from 0.1% to 5% by weight of the composition.

19. The composition of claim 13, further comprising a free zinc salt and wherein the free zinc salt ranges from 0.1% to 0.5% by weight of the composition.

20. The composition of claim 13, further comprising a fluoride compound.
